(19) United States
(12) Patent Application Publication
(10) Pub. No.: US 2009/0104242 A1
(43) Pub. Date: Apr. 23, 2009

(54) NIOBIUM OXIDE COMPOSITIONS, NANOSTRUCTURES, BIOACTIVE FORMS AND USES THEREOF

(75) Inventor: Robert L. Karlinsey, Indianapolis, IN (US)

Correspondence Address: BOSE MCKINNEY & EVANS LLP, 111 MONUMENT CIRCLE, SUITE 2700, INDIANAPOLIS, IN, 46204, US

(73) Assignee: INDIANA UNIVERSITY RESEARCH & TECHNOLOGY CORPORATI, Indianapolis, IN (US)

(21) Appl. No.: 11/997,096

(22) PCT Filed: Jul. 28, 2006
(86) PCT No.: PCT/US06/29336

§ 371 Date: Nov. 5, 2008

Related U.S. Application Data
(60) Provisional application No. 60/703,366, filed on Jul. 28, 2005.

(51) Int. Cl. A61F 2/04; C01G 33/00; A61K 33/24

(52) U.S. Cl. 424/423; 428/599; 423/594.17; 204/157.51; 424/600; 977/931

(57) ABSTRACT

Self-organized niobium oxide nanocones with nano-sized tips are prepared by anodization of niobium in the presence of an electrolyte such as hydrofluoric acid (HF) (aq.). Dimensions and integrity of the bulk nanostructures formed are strongly dependent on potential, temperature, electrolyte composition, and anodization times. Accordingly, the morphology, topology, uniformity and bioactivity of the niobium oxide nanostructures formed can be readily adjusted by adjusting these anodization parameters. A bioactive form of crystalline niobium oxide is formed by anodizing niobium metal in the presence of an electrolyte that includes HF and at least one salt such as Na₂SO₄ or NaF. One property of bioactive niobium oxide formed by anodizing niobium metal in the presence of HF (aq.) is its ability to interact with hydroxylapatite.

### PRIORITY CLAIM

This Application claims the benefit of U.S. Provisional Patent Application No. 60/703,366 filed on Jul. 28, 2005, which is incorporated herein by reference in its entirety.

### TECHNICAL FIELD OF THE INVENTION

The present invention relates to the formation and use of niobium oxides, including methods of forming crystalline niobium oxides with defined nanostructure morphologies features and/or with useful bioactivities.

### BACKGROUND

Niobium oxides were studied initially because of their utility in the construction of solid electrolyte capacitors [1] and superconductivity [2]. Recently, however, niobium oxide has commanded additional attention due to its promising potential in medical applications [3]. Perhaps, the most favorable form of niobium oxide in many applications is Nb₂O₅ due to its high resistivity to chemical attack, strong affinity to oxygen, carbon, and nitrogen, thermodynamic stability, and biocompatibility.

Typically, niobium oxide is formed through either a sol-gel process or electrochemical anodization. For further discussion please see, for example, [4,5].

Because of the great promise that niobium oxides have in applications ranging from electrical devices to medical implants there is a continued need for niobium oxides with useful properties and for methods for making niobium oxides. One aspect of the invention is to meet these needs.

One aspect is a material substantially comprising niobium oxide and having a well defined morphology and composition.

One embodiment is a self-organized composition including niobium oxide that can be prepared by potentiostatic anodization carried out in the presence of an electrolytic solution including an inorganic acid such as HF(aq).

Another embodiment is self-organized compositions of metal oxides formed by anodizing virtually any reactive metal or mixture thereof.

Still another embodiment is self-organized compositions of metal oxides formed by anodizing at least one metal selected from the group consisting of Al, Ti, and Zr in the presence of an electrolyte including, for example, dilute solutions of HF(aq).

In one embodiment the anodization is carried out in the presence of between about 0.25 wt. percent to about 10 wt. percent HF(aq.). In another embodiment the concentration of HF (aq.) is about 2.5 wt. percent. In still another embodiment HF (aq.) is supplement with another acid, for example, phosphoric acid.

Another embodiment is a method of forming niobium oxides that have a defined morphology and/or topology by anodizing niobium metal and controlling anodization parameters including electrolyte strength, voltage at constant potential, temperature. In one embodiment the electrolyte includes a salt that is soluble under the anodization conditions and that interacts with niobium metals example of suitable salts include, but are not limited to NaF and Na₂SO₄.

In one embodiment the anodization reaction of niobium metal to form niobium oxide is carried at a temperature range from about −10 degrees Celsius to about 110 degrees Celsius. In still another embodiment the anodization reaction of niobium metal to form niobium oxide is carried at a temperature range from about 20 degrees Celsius to about 110 degrees Celsius. In yet another embodiment the anodization reaction of niobium metal to form niobium oxide is carried at a temperature range from about 20 degrees Celsius to about 90 degrees Celsius. In still another embodiment the reaction is carried out at a temperature of about 22 degrees Celsius.

In one embodiment the anodization of niobium metal to form niobium oxide is carried out at a voltage in the range of between about 15 to about 150 volts. In still another embodiment the anodization reaction is carried out at voltage in the range of between about 15 to 100 volts. In yet another embodiment the anodization reaction is carried out at voltage in the range of between about 15 to 75 volts.

IN one embodiment niobium metal is anodized to niobium oxide in an electrolyte that includes a salt concentration of between about 10 mg of salt per 100 ml of electrolyte to about 350 mg of salt per 100 ml of electrolyte. In one embodiment the salt is selected from the group of salts consisting of NaF and Na₂SO₄. In still another embodiment additional or other salts that donate ions to niobium and are soluble in an electrolyte that includes HF(aq.) are present in the electrolyte.

Yet another embodiment includes coating a niobium oxide nanostructure with a metal or metal alloy, in one embodiment the nanostructures are coated with an alloy of gold and palladium (AuPd).

Still another embodiment includes using niobium oxide nanocones in the manufacture of filaments used to construct electrical devices, including but not limited to, photoelectric displays and imaging devices such as electron microscopes.

One embodiment is a bioactive crystalline niobium oxide formed by anodizing niobium metal in the presence of an electrolyte that includes sodium fluoride (NaF).

In one embodiment sodium fluoride levels used in the anodization process are between are between about 50 to about 500 mg per 100 mL of salt in the electrolyte. In still another embodiment the anodization is carried out in the presence of about 100 to about 200 mg of NaF per mL of salt in the electrolyte.

One embodiment includes using bioactive crystalline niobium oxides as coating for medical devices. Medical devices that can be coated with niobium oxide nanostructures made in accordance with various embodiments device include those that are intended for intimate contact with bone or tooth. Such devices include, but are not limited to screws, staples, pins, replacement parts, bands, plates, dolls, pegs, wires, bars, braces, rods, artificial joints, teeth, dentures, filings, bridges, crowns, caps and the like.

Another embodiment is a paste, liquid or coating including niobium oxides that are used to promote the healing and/or bonding of diseased, damaged, missing or malformed bone or teeth.

Still another embodiment includes a method of treating medical conditions, which implicate damaged, diseased or disfigured bone or teeth, by providing a suitable device which includes at least a coating of crystalline bioactive niobium oxide and placing the device in contact with tissues, fluids, sera, saliva or synthetic mimics thereof that induce the development of hydroxyapatite (HAP).

Yet another embodiment is a bioactive crystalline niobium oxide surface that accommodates HAP formation when contacted with a mucin-containing acellular simulated bodily fluid.

Still another embodiment is to add niobium oxide nanostructures to various dentifrices and other preparations for dental treatments. Formalizations or oral care and/or treatment that can niobium oxides include, but are not limited to, desensitizers, preparation that treat sensitive teeth, by for example augmenting dentin tubules in the process of dentition of teeth that are sensitive to stimuli such as changes or extremes in temperatures and materials rich in sugar, salt or acid. The niobium oxide nanostructures can be admixed with suitable surfactants such as aliphatic alcohols and or polyethylene glycol or biocompatible polymers such as polycaprolacton in various dentifrices for delivery of the oxide to various HAP rich components in the oral cavity.

In yet another embodiment, bioactive niobium oxides are added to glues, cements, grouts, fillings and the like for use in repairing damaged, diseased, malformed or missing bones or teeth.

Another embodiment is the use of niobium oxide nanostructures made in accordance with some embodiments in the construction of sensors. The nanostructures can be used to interact with various components in a sample of either gas or liquid or the niobium oxide nanostructures can be coated with material that selectively or at least differentially interacts with a least one compound in the sample. In one embodiment this interaction generates a signal and the sensor can be used to detect either the presence of absence of a given compound in a given sample.

In one embodiment the nanostructures are used in the manufacture of sensors for detecting and or measuring the presence of DNA, RNA or other molecules in a sample. In one embodiment the niobium oxide nanostructures are coated with a precious metals such as platinum, palladium rhodium, ruthenium, iridium, gold, silver, rhenium, osmium, nickel, copper, zinc and alloys of these and other metals and/or some oxides that selectively interacts with a least one compound in a sample.

In still another embodiment niobium oxide nanostructures are coated with a catalytic material and used to catalyze at least one chemical reaction. Catalytic materials that can be applied to the niobium oxide nanostructures include, but are not limited to, precious metals such as platinum, palladium rhodium, ruthenium, iridium, gold, silver, rhenium, osmium, nickel, copper, zinc and alloys of these and other metals and/or some oxides.

In one embodiment niobium oxide nanostructures are used to construct sensors that include at least one antibody.

In another embodiment niobium oxide nanostructures are used to construct sensors that include at least one molecule that changes fluorescence when the molecule contacts a nucleic acid polymer such as DNA or RNA.

In still another embodiment niobium oxide nanostructures are used to construct sensors that include at least one molecule that changes fluorescence when the molecule contacts a nucleic acid polymer such as DNA or RNA which as been tagged or labeled with a molecule that selectively or preferentially binds to the fluorescent molecule.

In one embodiment niobium oxide nanostructures are used to construct sensors for the detection and/or measurement of biomolecules such as nucleic acids, peptides, polypeptides, amino acids, sugars, polysaccarides, fatty acids, hormones, growth factors, signaling molecules, neurotransmitters, and antibodies.

In another embodiment niobium oxide nanostructures are used to construct sensors for the detection and/or measurement of specific organic or inorganic compounds or specific classes of organic or inorganic compounds.

In another embodiment niobium oxide nanostructures are used to construct sensors that selectively detect and/or bind at least one pathogen selected from the group consisting of bacteria, molds, fungi, viruses and protozoa.

Another embodiment is a niobium oxide nanostructure used to construct device for the separation of various components in a liquid or gas sample.

In one embodiment niobium oxide nanostructures either by themselves or suitably derivative or coated can be used to create chromatographic columns for use in either liquid of gas chromatography. In one embodiment these chromatographic devices are designed to separate at least one component from samples that include mixtures of compounds. Depending on the selectivity of the material used to coat the nanostructures these devices can be used to separate mixtures of biomolecules, organic molecules, inorganic molecules and/or combination of all of the above.

One embodiment is a chromatography device including a niobium oxide nanostructures include coated with a compound that selectively or differentially interacts with at least one component in a mixture. Depending on the materials to be separated the coatings can include precious metals such as platinum, palladium rhodium, ruthenium, iridium, gold, silver, rhenium, osmium, nickel, copper, zinc and alloys of these and other metals and/or some oxides. In still another embodiment the nanostructures are coated with antibodies, polymers, nucleic acid polymers and the like in order to form devices suitable for separating components of various mixtures.

### BRIEF DESCRIPTION OF THE FIGURES

FIG. 1. A schematic illustrating one apparatus used to make a compound comprising niobium oxide through anodization.

FIG. 2. Energy Dispersive Spectra showing a material comprising niobium.

FIG. 3. Energy Dispersive Spectra of a material comprising niobium.

FIG. 4. A SEM image; top views of a niobium oxide nanostructure formed by anodizing niobium metal 7.5 hours in an electrolyte including about 1.5 wt. % HF(aq) at 22 degrees C., under the following constant potentials; 25 volts panel (A), 40 volts panel (B), 30 volts panel (C), and 90 volts panel (D).

FIG. 5. A Scanning Electron Microscope (SEM) image; cross-sectional view of a niobium oxide nanostructure formed by anodizing niobium metal under about 25 volts for about 0.5 hours in an electrolyte including about 2.5 wt. % HF(aq).

FIG. 6. A SEM image; cross-sectional view of a niobium oxide nanostructures formed by anodizing niobium metal under about 25 volts for about 2.0 hours in an electrolyte including about 2.5 wt. % HF(aq).

FIG. 7. A SEM image; cross-sectional view of a niobium oxide nanostructure formed by anodizing niobium metal under about 25 volts in an electrolyte including about 1.5 wt. % HF(aq) at room temperature.

FIG. 8. SEM images; side-views of a niobium oxide nanostructure formed by anodizing niobium metal under about 25 volts at room temperature in an electrolyte including about 2.5 wt. % HF(aq); (A) the side of a conical nanostructure and (B) the top of the conical nanostructures.

FIG. 9. SEM images; top view showing the growth of niobium oxide nanostructures formed by anodization. The nanostructures were formed under about 25 volts at room temperature in an electrolyte including about 1.5 wt. % HF(aq) for; (A) 2 hours, (B) 3 hours; (C) 4 hours; and (D) 6.5 hours.

FIG. 10. A SEM image; cross-sectional views illustrating “growth rings” in a niobium oxide micro-nanostructure formed by anodizing niobium metal under about 15 volts under room temperature in an electrolyte including about 1.5 wt. % HF(aq). FIG. 10(A) an image collected at a relatively low magnification 10(B) and image collected twice the magnification used to collect the image in FIG. 10(a).

FIG. 11. A SEM image; top views of a niobium oxide nanostructures formed by anodizing niobium metal an electrolyte solution including 1.5 wt. % HF, at room temperature. The material shown in panel A was formed t a constant potential of 30 V and the material shown in panel (B) was formed at 40 volts.

FIG. 12. X-Ray Diffraction (XRD) pattern of a crystalline niobium oxide film formed by anodizing Nb metal in the presence of NaF. The oxide was soaked for 16 hours in artificial saliva and this pattern was collected. Features of the pattern include a pronounced crystal nanostructure belonging to Nb₂O₅ when indexed (JCPDS# 30-0873) and Hydroxylapatite (HAP) formation (JCPDS #09-0432) shown marked with an asterisk.

FIG. 13. X-Ray Diffraction patterns of niobium oxides formed by anodizing Nb metal and then soaking the material in artificial saliva before collecting the patterns. The pattern shown with double lines is of an oxide formed in the presence of NaF; the pattern shown in the solid line was formed in the absence of added NaF. Only the pattern with the double line shows a feature, marked with an asterisk that indexes with (HAP).

FIG. 14. SEM images of niobium oxide crystals in contact with hydroxyapitie (HAP); (A) image collected at a relatively low magnification (B) image collected relatively high magnification.

FIG. 15. Schematic diagrams illustrating elements of (A) an electron gun and (B) an electron microscope including an electron gun.

### DETAILED DESCRIPTION

For the purposes of promoting an understanding of the principles of the invention, reference will now be made to the embodiments illustrated herein and specific language will be used to describe the same. It will nevertheless be understood that no limitation of the scope of the invention is thereby intended. Any alterations and further modifications in the described processes, systems or devices, and any further applications of the principles of the invention as described herein, are contemplated as would normally occur to one skilled in the art to which the invention relates.

Most terms are given their usual and customary meaning as used in the art to which the various embodiments are directed. Some terms are clarified as follows. As used herein the terms “pharmaceutically-acceptable topical oral carrier,” or “topical, oral carrier,” generally means one or more compatible solid or liquid fillers, diluents or encapsulating substances that are suitable for topical, oral administration. The term, “compatible,” as used herein, means that components of the composition are capable of being commingled without interacting in a manner which would substantially reduce the composition's stability and/or efficacy for treating or preventing oral care conditions such as caries, according to the compositions and methods of the present invention.

The term “about” generally refers to range of plus or minus on the order of ten percent of the value the entire range being on the order of 20 percent of the relevant value.

A therapeutically effective dosage or amount of a compound is an amount sufficient to affect a positive effect on a given medical condition. The affect if not immediately may, over period of time, provide a noticeable or measurable effect on a patient's health and well being.

Unless it specially states otherwise the terms ‘structures,’ ‘nanocones,’ ‘nanostructures’ and ‘microstructures’ used to describe niobium oxide formed by anodizing niobium metal in various embodiments of the invention are used interchangeably.

A number of explanations and experiments are provided by way of explanation and not limitation. No theory of how the invention operates is to be considered limiting whether proffered by virtue of description, comparison, explanation or example.

With the possible exception of gold, the formation of oxides on metals is omnipresent under standard temperature and pressure in the presence of oxygen. A number of studies have been reported elucidating the preparation and utility of novel nanoporous metal oxide nanostructures for applications including catalysis, sensing, and bio-engineering see, for example, [6-7].

Some of these studies report the formation of metal oxide nanostructures that have two- and three-dimensional geometries including pores [9] and tubes, [10]. These nanostructures may be developed in several ways including templating [11], anodization [12], and sol-gel processes [3]. In terms of cost, purity, and convenience anodization offers a particularly attractive means for producing useful metal oxides.

The most popular oxides used to form structures that have a defined shape include oxides of aluminum and titanium [11, 10]. These particular oxides have attracted a lot of interest, in part because; they are relatively easy to prepare. However, oxides of other metals, such as niobium, are also of interest because they may have certain advantageous over other more commonly used metal oxides.

Niobium oxide in particular may be of considerable utility because of its extremely high corrosion resistance and thermodynamic stability. These properties render niobium oxide a promising candidate for use in, for example, coatings for improved osteoblast cell adhesion on artificial implants or for use in electronic, electrochromic, ferroelectric devices, sensors and separation columns sand devices. For additional general discussion of these applications please see [1, 3, 13].

Despite considerable research on the formation mechanism, composition, and uses of metal oxides, relatively little has been reported on the self-organized morphologies of metal oxides in general and on niobium oxides in particular [2]. Some recent studies report the preparation of nanoporous niobium oxide structures. For a more extensive discussion of metal oxide nanoporous structures the reader is directed to [5, 13].

The lack of morphological options in forming and shaping metal oxides such as niobium oxide is impeding the use and development of metal oxides in promising material science and medical applications. One aspect of the invention provides methods for forming self-organized niobium oxide nanostructures.

One embodiment includes a nano-tipped niobium oxide nanocones prepared via electrochemical anodization carried out in the presence of an electrolyte including an inorganic acid. One inorganic acid useful as an electrolyte in this process is HF.

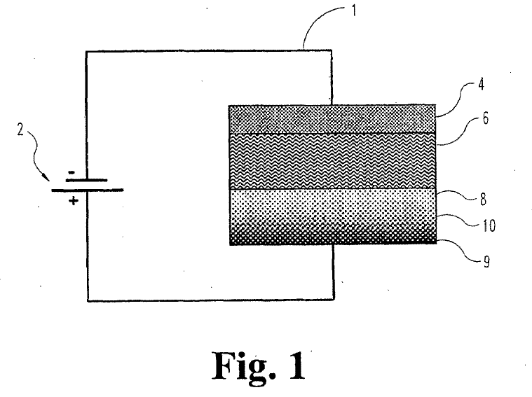

Referring now to FIG. 1, a schematic diagram of an anodization set-up (1) that can be used to produce various niobium oxides in accordance with some embodiments of the invention. Device (1) includes: a power source (2); a layer of copper metal (4) an electrolyte (6) a layer of niobium metal (10). As the reaction proceeds a layer of niobium oxide (8) accumulates on the surface of metal (9).

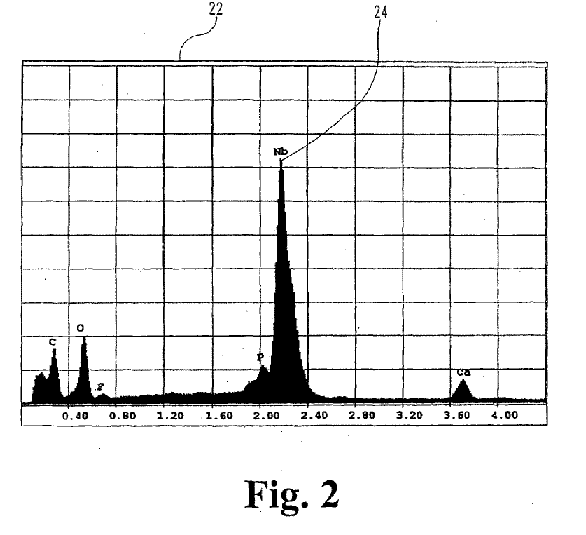

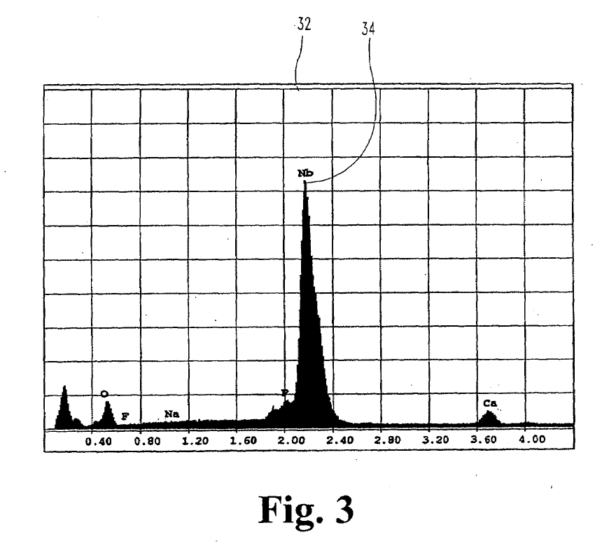

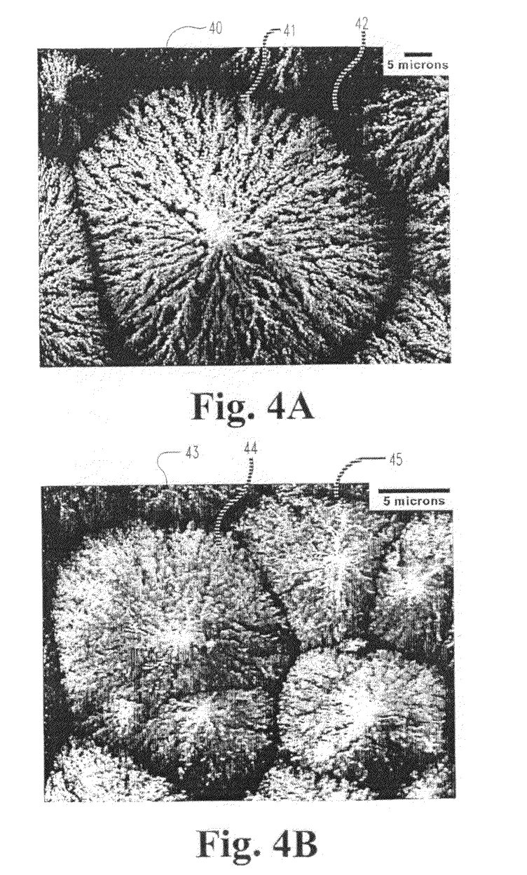

Referring now to FIGS. 2 and 3; both show Energy Dispersion Spectra of materials, which include niobium. These materials were formed by anodization of niobium carried out at a constant potential.

Referring now to FIG. 2, the material analyzed in FIG. 2 was formed by anodizing niobium metal for 68 min. at 20 volts, 46 degrees C. in an electrolyte that included 100 mg of NaF per 100 mL of 2.5 wt. % HF(aq). This spectrum (22) shows a very distinct peak (24) identified as niobium.

The material analyzed in FIG. 3 was formed by anodizing niobium metal for 90 min. at 20 volts, 50 degrees C. in an electrolyte that included 200 mg of NaF per 100 mL of 2.5 wt. % HF(aq). This spectrum (32) shows a very distinct peak (34) identified as niobium.

Still another embodiment includes niobium oxide nanostructures formed under anodization conditions including varying concentration of HF(aq), the presence and absence of NaF, different anodizing times, different temperatures, and electrical potentials.

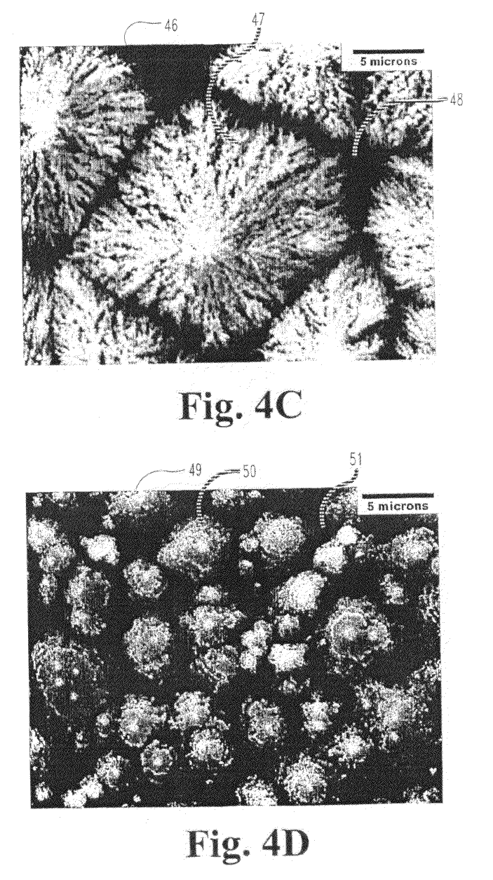

Referring now to FIG. 4; top views of one embodiment niobium oxide nanostructures formed by anodizing niobium metal. All of the nanostructures shown in panels (A) through (D) (40), (43), (46) and (49) respectively were formed by anodization carried out at 22 degrees C., in 1.5 wt. % HF(aq). All showed distinct peaks (41), (44), (47) and (50); and gaps (42), (45), (48), (51) between peaks (41), (44), (47) and (50). All niobium oxide microstructures shown in FIG. 4 were formed at different constant voltages: those in panel (A) were formed at 25 volts; those in panel (B) were formed at 40 volts; those in panel (C) were formed at 30 volts; and those in panel (D) were formed at 90 volts. These data indicate that, other parameters held equal, the size of the niobium nanocones formed varies with the voltage used.

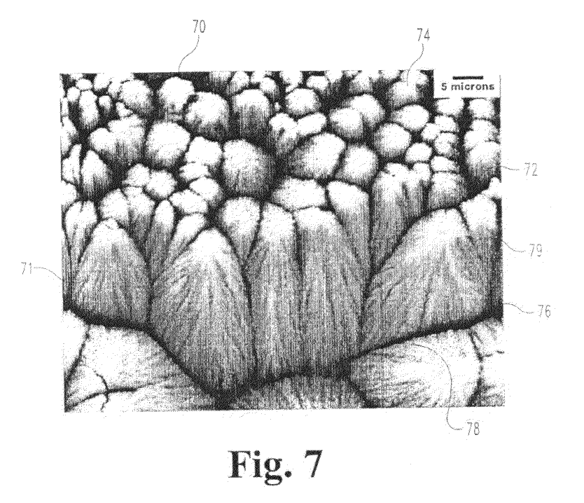

Referring now to FIG. 7; a SEM image (70) a cross-sectional view of niobium oxide nanocone structures (71) formed by anodizing niobium metal. These nanostructures (71) were formed by anodizing niobium metal at a constant potential of 25 volts, at room temperature, in the presence of an electrolyte that includes 2.5 wt. % HF. Microstructures (71) are in the generally shape of a nanocone and have: distinct tops (74); sides (72), a common base (78); and crevices (78) between individual nanocones (71).

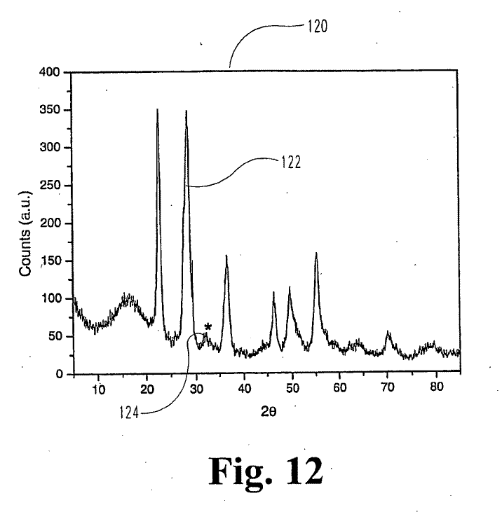

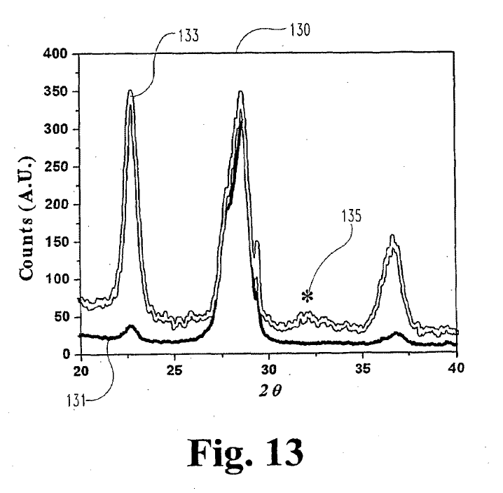

Another embodiment is the use of bioactive niobium oxides in a variety of medical applications. As 5 illustrated in FIGS. 12, 13 crystalline niobium oxides formed in the presence of NaF can bind to hydroxyapatite (HAP). These patterns show a feature (marked with an asterisk) that is indicative of HAP when indexed it match with (JCPDS #09-0432).

Bioactive niobium oxides made in accordance with various embodiments of the invention interacts with hydroxylapatite. Hydroxylapatite is found in human and animal, bone, teeth, tooth enamel, and dentin. One form of hydroxylapatite is represented by the formula Ca₅(PO₄)₃(OH) sometimes written as Ca₁₀(PO₄)₆(OH)₂.

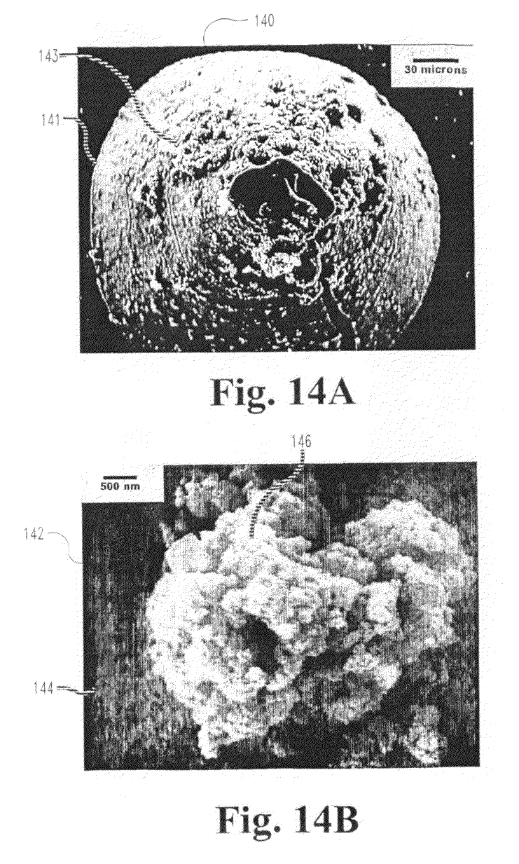

Referring now to FIG. 14, additional evidence of crystalline niobium oxide binding with HAP is shown in SEM images 141 and 144. Referring now to FIG. 14 (A) crystalline niobium oxide microcone 141 shown in SEM image 140 was formed by anodizing niobium metal for 90 min. under 20 volts at 50 degrees C. in the presence of an electrolyte comprising 200 mg per mL of NaF in 2.5 wt. % HF (aq). Before image 140 was taken, the material was immersed in artificial saliva for 19 hours. This induced the formation of HAP crystal (143) on the niobium oxide crystal nanostructure (141).

Referring now to FIG. 14 (b); SEM image (142). Crystalline niobium oxide microcone (144) was formed by anodizing niobium metal for 2.5 hours under 20 volts at 46 degrees C. in the presence of an electrolyte comprising 100 mg per mL NaF in 2.5 wt. % HF (aq). Before image (140) was taken the material was immersed in artificial saliva for 19 hours. This induced the formation of HAP crystal (146) on the niobium oxide crystal structure (144).

As illustrated in SEM images FIGS. 4-11 various niobium oxides made in accordance with a number of embodiments have a rough surface. This rough surface makes for a large surface area and when combined with the material's affinity for hydroxylapatite (HAP) implies utility as an interface between teeth, bone and artificial materials that are intended to interact strongly with teeth and bone and the like. Still another embodiment is using of bioactive crystalline niobium oxides to mend, support, shape, knit, or replace elements of bone, teeth and similar tissues in human and animal patients.

The shape and size of the nanostructures formed can be readily adjusted by varying the anodization parameters, such as the thickness of niobium metal starting material. To a first approximation the thicker the metal to begin with the higher the conical structure that can be formed via the anodization process. Voltage values range of between 15 to about 150 volts can be used. Other useful ranges include values of between about 15 to about 100 and between about 15 to about 75 volts.

Temperature also affects that rate of oxide formation and to some extent the shape of the nanostructures. Suitable temperatures for carrying out the anodization reaction range from about −10 degrees Celsius to about 110 degrees Celsius, other suitable ranges include from about to 20 degrees Celsius about 10 degrees Celsius and from about 20 degrees Celsius to about 90 degrees Celsius.

The anodization reaction can be carried out so long as there is niobium metal to be oxidized. While the reaction, given sufficient metal, has the potential to run for days as a practical matter various assays conditions will likely be adjusted to form suitable nanostructures in a matter hours.

Anodization of Niobium metal to form bioactive niobium oxides according to various embodiments of the invention generally include HF(aq.) in the electrolyte. In some embodiments additional acids may be added to HF (aq.), including, for example, phosphoric acid.

The amount and composition of electrolyte also influences the size and shape of the nanostructure formed. Bioactive niobium oxides are formed in the presence of hydrofluoric acid (HF). Suitable ranges of HF(aq.) for the process range from about 1 wt. percent to about 15, wt. percent, other useful ranges for HF include about 2.5 to about 10.0 wt. percent, in one embodiment the concentration of HF(aq.) in the reaction is on the order of about 2.5 wt. percent.

The level of salt added to the electrolyte also influences the rate of the reaction and the shape of the nanostructures. Any salt with the capacity to contribute ions to the niobium metal layer and that is soluble in HF(aq.) can be used in the electrolyte. Typical salts used include HF and Na₂SO₄.

One embodiment includes stabilizing the otherwise fragile niobium oxide nanostructures by covering them with less brittle materials such as silver, copper or of alloys of gold and palladium (AuPd). Additional metals that can be used to coat niobium oxide nanostructure include, but are not limited to, gold, platinum, palladium, ruthenium, rhodium, iridium, silver, rhenium, osmium, nickel, copper, zinc and alloys thereof.

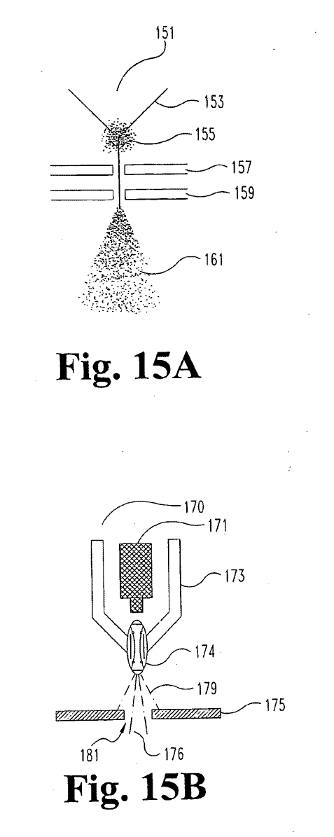

Still another embodiment includes using these niobium oxide nanocones in the manufacture of electrical devices. Devices that may benefit from the use of such fine tipped nanostructure include but are not limited to devices illustrated schematically in FIG. 15.

FIG. 15 (A) shows an electron gun (151) that can be used in photoelectric displays that are used in photoelectric displays. A typical electron gun of this form includes: a filament (153); a cathode (157); an anode (159); current through the filament (153) creates an electron cloud (155) directly above a gap between cathode (157) and anode (159). The effect of this gap is to accelerate and focus the electrons in cloud (155) to from the spray of electrons (161).

Additional uses for niobium oxide conical microstructures formed according to various embodiments include using them in the manufacture of devices for focusing electron beams in analytical instruments. Such instruments include, but are not limited to, electron microscopes such as scanning electron microscopes.

Referring now to FIG. 15 (B) a schematic representation of an electron focusing device (170) used in an electron microscope. Various parts include: a filament (171); a source of negative potential referred to a Wehnelt Cap (173); a space charge (174); an anode plate (175). Briefly, an electrical charge to filament (171) produces a stream of electrons that are focused by a gap (177) in Wehnelt Cap (173); this produces a beam of electrons (179) which is accelerated towards a gap (181) in anode plate (175).

Referring still to FIG. 15(B) the resolution of these types of devices is at least in part dependent upon the fineness of the electrical stream which is in turn at least partially dependent upon the filament (171) used to construct the electron gun (170). Accordingly, nano-tipped, conical nanostructures comprising niobium oxide nanostructures can be used to build electron microscopes with very high resolution.

Still another use for these nanostructures is as filaments in the construction of high resolution photo-electronic displays.

Another embodiment is to use niobium oxide nanostructures in the construction of sensors. The nanostructures can be coated with various materials that selectively interact with at least one component of a mixture of gasses or liquids. As samples are placed in contact with the surface a signal is generated when at least one component in the sample interacts with the surface of the sensor. Suitable coating depending upon the analyte include metals such as platinum, palladium rhodium, ruthenium, iridium, gold, silver, rhenium, osmium, nickel, copper, zinc and alloys of these and other metals as well as oxides of the same.

In still another embodiment niobium oxide nanostructures are coated with materials that selectively interact with specific organisms or components of organisms. In one embodiment the nanostructure may be coated with materials that selectively interact with structures on the surface of pathogenic bacteria, virus, molds, fungi, protozoa and the like.

In one embodiment the surface is coated with molecules that hybridize either directly or indirectly with nucleic acid polymers such as DNA or RNA. Direct binding can be accomplished by coating the surface of the nanostructure with segments of nucleic acid polymer that are complimentary to target sequences in a given sample, under hybridize to at least one DNA or RNA sequence in the sample under a given set of assay conditions. Indirect binding may be accomplished by coating the surface of the sensor with a material that preferentially binds to tags or labels placed attached to at least one nucleic acid polymer in the sample. In one embodiment niobium oxide nanostructures are coated with at least molecule that exhibits a change in fluorescence when it interacts with a given sequence of a nucleic acid polymer such as DNA and/or RNA.

In still another embodiment the nanostructures of niobium oxide are coated with materials that selectively or preferentially interact with biomolecues such as amino acids, peptides, polypeptides, proteins, sugars, polysaccharides, nucleic acids, signally molecules, neurotransmitters, hormones, fatty acids, alcohols, antibodies and the like.

In still another embodiment the surface is coated with materials that selectively interact with various, metals, metal alloys, metal oxides, other inorganic molecules and organic molecules.

In another embodiment niobium oxide nanostructures used in the construction of devices used in chromatography, the separation of components of various mixtures based on their physical and or chemical properties. Such devices include, but are not limited to, gas chromatography can liquid chromatography columns. The devices can be comprised of niobium oxide nanostructures that provide a large surface area and interact with component of a given gas or liquid sample. In still another embodiment the nanostructures are coated with materials that differentially or selectively interact with at least one component of a mixture of compounds in a given sample. Various coatings include, but are not limited to, metals, metal oxides, antibodies, and the like.

Metals, metal alloys and some metal oxides may be applied to the surface of the niobium nanostructures by techniques including, but not limited to, sputtering, electron spray, electron laser desorption, and electrolysis.

In still another embodiment niobium oxide nanostructures are used in the construction of catalysts. In some embodiment the surface of the nanostructure is coated with a metal or mixture of metals that catalyze various reactions. Metal suitable for this use include, but are not limited to, platinum, palladium, rhodium, ruthenium, iridium, gold, silver, rhenium, osmium, nickel, copper, zinc and alloys of these and other metals as well as some oxides of the same.

As illustrated in various examples throughout the application, the bioactive niobium oxide nanostructures disclosed in various embodiments also readily interacts with hydroxylapatite, (HAP) a fundamental component in the construction of human teeth and bones.

Niobium oxide nanostructures according to these embodiments may be added to various preparations for use in the care and treatment of teeth and bones in the oral cavity. For example, they may be added to desensitizers wherein their ability to bind to teeth and hydroxylapatite (HAP) in the presence of saliva can be used to treat teeth which are exceptionally sensitive to various chemicals and sensations including, for example, temperature, sweetness, etc.

In another embodiment, bioactive niobium oxides of some embodiments are incorporated into dentifrices in the form of a gel, paste, strip, rinse, gum or varnish; typically the oxide is admixed with various suitable dental surfactants. Various components of dental surfactants and other dentifrices that can be used in combination with niobium oxide microstructures of various embodiments are as follows.

The carriers of the present invention may include the usual and conventional components of toothpastes (including gels and gels for subgingival application), mouth rinses, mouth sprays, and more many of these are more fully described, hereinafter.

The choice of a carrier to be used is generally determined by the way the composition is to be S introduced into the oral cavity. If a tooth paste (including tooth gels, etc.) is to be used, then a “toothpaste carrier” is chosen and may include for, example, abrasive materials, sudsing agents, binders, humectants, flavoring and sweetening agents and the like as disclosed in, for example, U.S. Pat. No. 3,988,433, to Benedict, issued on Oct. 25, 1976, which is incorporated herein by reference. If a mouth rinse is to be used, then a “mouth rinse carrier” is chosen, such as water, flavoring and sweetening agents as disclosed in, for example, U.S. Pat. No. 3,988,433 issued to Benedict, and incorporated herein by reference in its entirety. Similarly, if a mouth spray is to be used, then a “mouth spray carrier” is chosen. If a sachet is to be used, then a “sachet carrier” is chosen (e.g., sachet bag, flavoring and sweetening agents). If a subgingival gel is to be used (for delivery of the active material into the periodontal pockets, or around the periodontal pockets, then the material may be combined with a, “subgingival gel carrier”. Suitable subgingival carries include those disclosed in U.S. Pat. No. 5,198,220, Damani, issued Mar. 30, 1993, P&G, U.S. Pat. No. 5,242,910, Damani, issued Sep. 7, 1993, all of which are incorporated herein by reference in their entirety. Carriers suitable for the preparation of compositions of the present invention are well known in the art. Their selection will depend on secondary considerations such as mouth feel, taste, cost, shelf stability and the like.

Preferred compositions for use in various embodiments may be in the form of dentifrices, such as toothpastes, tooth gels, tooth polishes and tooth powders. Components of such toothpaste and tooth gels generally include one or more of a dental abrasive (from about 10% to about 50%), a surfactant (from about 0.5% to about 10%), a thickening agent (from about 0.1% to about 5%), a humectant (from about 10% to about 55%), a flavoring agent (from about 0.04% to about 2%), a sweetening agent (from about 0.1% to about 3%), a coloring agent (from about 0.01% to about 0.5%) and water (from about 2% to about 45%). Such toothpaste or tooth gel may also include one or more of an additional anticaries agent (from about 0.05% to about 10% additional anticaries agent), and an anticalculus agent (from about 0.1% to about 13%). Tooth powders, of course, contain substantially all non-liquid components.

Other preferred compositions for use in various embodiments include, for example, non-abrasive gels, including subgingival gels. Gel compositions commonly include a thickening agent (from about 0.1% to about 20%), a humectant (from about 10% to about 55%), a flavoring agent (from about 0.04% to about 2%), a sweetening agent (from about 0.1% to about 3%), a coloring agent (from about 0.01% to about 0.5%), water (from about 2% to about 45%), and may comprise an additional anticaries agent (from about 0.05% to about 10% of additional anticaries agent), and an anticalculus agent (from about 0.1% to about 13%).

Other preferred compositions for use in various embodiments may include, for example, mouthwashes, mouth rinses, and mouth sprays. Components of such mouthwashes and mouth sprays typically include one or more of water (from about 45% to about 95%), ethanol (from about 0% to about 25%), a humectant (from about 0% to about 50%), a surfactant (from about 0.01% to about 7%), a flavoring agent (from about 0.04% to about 2%), a sweetening agent (from about 0.1% to about 3%), and a coloring agent (from about 0.001% to about 0.5%). Such mouthwashes and mouth sprays may also include one or more additional anticaries agents present, for example, from about 0.05% to about of additional anticaries agent, and an anticalculus agent present, for example, from about 0.1% to about 13%.

Other preferred compositions for use with various embodiments include, for example, dental solutions. Components of such dental solutions generally may include one or more of water present from about 90% to about 99%, preservative present from about 0.01% to about 0.5%, thickening agent present from 0% to about 5%, flavoring agent present from about 0.04% to about 2%, sweetening agent present from about 0.1% to about 3%, and surfactant present in such compositions from about 0% to about 5%.

Types of carriers which may be included in compositions of the present invention, along with specific non-limiting examples, abrasives, sudsing agents many of which are surfactants, thickening agents, humectants, flavoring and sweetening agents, anticalculus agents, alkali metal bicarbonate salts, and miscellaneous carriers.

Dental abrasives useful in the topical, oral carriers of the compositions of various embodiments include many different materials. Various suitable materials are preferably materials that are compatible within the composition of interest and one that do not excessively abrade dentin. Suitable abrasive materials include, for example, silicas including gels and precipitates, insoluble sodium polymetaphosphate, hydrated alumina, calcium carbonate, dicalcium orthophosphate dihydrate, calcium pyrophosphate, tricalcium phosphate, calcium polymetaphosphate, and resinous abrasive materials such as particulate condensation products of urea and formaldehyde.

Another class of abrasives for use in various embodiments include, for example, particulate thermo-setting polymerized resins as described in U.S. Pat. No. 3,070,510 issued to Cooley & Grabenstetter on Dec. 25, 1962. Suitable resins include, for example, melamines, phenolics, ureas, melamine-ureas, melamine-formaldehydes, urea formaldehyde, melamine-urea-formaldehydes, cross-linked epoxides, and cross-linked polyesters. Various mixtures of various abrasives may also be used.

Silica dental abrasives of various types may be used in some embodiments because they provide exceptional dental cleaning and polishing performance without unduly abrading tooth enamel or dentine. The silica abrasive polishing materials described herein, as well as other abrasives, generally have an average particle size ranging between about 0.1 to about 30 microns, and preferably from about 5 to about 15 microns although materials with differing sizes may also be used in various embodiments. The abrasive can be precipitated silica or silica gels such as the silica xerogels described in U.S. Pat. No. 3,538,230 issued to Pader et al., on Mar. 2, 1970, and, U.S. Pat. No. 3,862,307, issued to DiGiulio on Jan. 21, 1975, both of which incorporated herein by reference in their entirety. Preferred are the silica xerogels marketed under the trade name “Syloid” by the W.R. Grace & Company, Davison Chemical Division. Also preferred are the precipitated silica materials such as those marketed by the J. M. Huber Corporation under the trade name, Zeodent®, particularly the silica carrying the designation Zeodent 119®. For a more thorough discussion and listing of types of silica dental abrasives useful in the toothpastes the reader is directed to see, U.S. Pat. No. 4,340,583, issued to Wason on Jul. 29, 1982, and incorporated herein by reference in its entirety. The abrasive in the toothpaste compositions described herein is generally present at a level of from about 6% to about 70% by weight of the composition. Preferably, toothpastes may contain from about 10% to about 50% of abrasive, by weight of the composition.

One type of precipitated silica for use in various embodiments is disclosed in U.S. Pat. No. 5,603,920, issued on Feb. 18, 1997; U.S. Pat. No. 5,589,160, issued Dec. 31, 1996; U.S. Pat. No. 5,658,553, issued Aug. 19, 1997; U.S. Pat. No. 5,651,958, issued Jul. 29, 1997, all of which incorporated herein by reference in their entirety.

A variety of mixtures of abrasives can also be used. All of the above patents regarding dental abrasives are incorporated herein by reference. The total amount of abrasive in dentifrice compositions in various embodiments may generally range from about 6% to about 70% by weight; commonly toothpastes contain from about 10% to about 50% of abrasives, by weight of the composition. Solution, mouth spray, mouthwash and non-abrasive gel compositions of the subject invention typically contain no abrasive, although abrasive materials may be added to such compositions.

Suitable for use in various embodiments include sudsing agents that are reasonably stable and form foam throughout a wide pH range. Sudsing agents include, but are not limited to, nonionic, anionic, amphoteric, cationic, zwitterionic, synthetic detergents, and mixtures thereof. Many suitable nonionic and amphoteric surfactants are disclosed in U.S. Pat. No. 3,988,433 issued to Benedict on Oct. 26, 1976 and U.S. Pat. No. 4,051,234, issued to Gieske et al. on Sep. 27, 1977. Many suitable nonionic surfactants are disclosed by Agricola et al., U.S. Pat. No. 3,959,458 to Agicola et al., issued on May 25, 1976, both of which are incorporated herein by reference in their entirety.

Various nonionic and amphoteric surfactants may be used in various embodiments. As used herein, nonionic surfactants that may be used in various embodiments can be broadly defined as compounds produced by the condensation of alkylene oxide groups (hydrophilic in nature) with an organic hydrophobic compound which may be aliphatic or alkyl-aromatic in nature. Examples of suitable nonionic surfactants include, but are not limited to, poloxamers (sold under trade name Pluronic), polyoxyethylene sorbitan esters (sold under trade name Tweens), fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, and mixtures of such materials.

As used herein various amphoteric surfactants that can be used in various embodiments can be broadly described as derivatives of aliphatic secondary and tertiary amines in which the aliphatic radical can be a straight chain or branched and wherein one of the aliphatic substituents contains from about 8 to about 18 carbon atoms and one contains an anionic water-solubilizing group, e.g., carboxylate, sulfonate, sulfate, phosphate, or phosphonate. Other suitable amphoteric surfactants are betaines, specifically cocamidopropyl betaine. Mixtures of amphoteric surfactants can also be used in various embodiments.

Various embodiments may typically comprise a nonionic, amphoteric, or combination of nonionic and amphoteric surfactant each at a level of from about 0.025% to about 5%, in another embodiment from about 0.05% to about 4%, and in even another embodiment from about 0.1% to about 3% by weight, although other ranges of such materials may be present in various embodiments.

As used herein, anionic surfactants that can be added to various embodiments include water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate and sodium coconut monoglyceride sulfonates are examples of anionic surfactants of this type. Other suitable anionic surfactants are sarcosinates, such as sodium lauroyl sarcosinate, taurates, sodium lauryl sulfoacetate, sodium lauroyl isethionate, sodium laureth carboxylate, and sodium dodecyl benzenesulfonate. Various mixtures of anionic surfactants can also be employed. Some embodiments typically comprise an anionic surfactant at a level of from about 0.025% to about 9%, and in another embodiment from about 0.05% to about 7%, and in still another embodiment from about 0.1% to about 5% by weight.

Toothpastes and gels typically include a thickening agent added to the compound to create a desirable consistency, to provide desirable release characteristics when used, to increase shelf stability, and to increase the overall stability of the composition, etc. Preferred thickening agents that may be used in various embodiments include, but are not limited to, carboxyvinyl polymers, carrageenan, hydroxyethyl cellulose, laponite and water soluble salts of cellulose ethers such as sodium carboxymethylcellulose and sodium carboxymethyl hydroxyethyl cellulose. Natural gums such as gum karaya, xanthan gum, gum arabic, and gum tragacanth can also be used. Colloidal magnesium aluminum silicate or finely divided silica may be added to further improve the texture of the composition.

Thickening agents may include, with the exception of polymeric polyether compounds, e.g., polyethylene or polypropylene oxide (M.W. 300 to 1,000,000), capped with alkyl or acyl groups containing 1 to about 18 carbon atoms.

A preferred class of thickening or gelling agents for use in various embodiments includes a class of homopolymers of acrylic acid cross linked with an alkyl ether of pentaerythritol or an alkyl ether of sucrose, or carbomers. Carbomers are commercially available from B. F. Goodrich as the Carbopol™ series. Additional carbopols that may be included in various embodiments includes Carbopol 934, 940, 941, 956, and mixtures thereof.

Subgingival gel carrier for use in or around periodontal pockets periodontal pockets may include copolymers of lactide and glycolide monomers. A typical copolymer for use in these compositions has a molecular weight in the range of from about 1,000 to about 120,000 these values are average numbers for the molecular weights of the various components. For a more through discussion and listing of such polymers the reader is directed to see: U.S. Pat. No. 5,198,220, issued to Damani, on Mar. 30, 1993; U.S. Pat. No. 5,242,910, issued to Damani, on Sep. 7, 1993; and U.S. Pat. No. 4,443,430, issued to Mattei, on Apr. 17, 1984, all of which are incorporated herein by reference in their entirety.

Thickening agents in an amount from about 0.1% to about 15%, or from about 0.2% to about 6%, in another embodiment from about 0.4% to about 5%, by weight of the total toothpaste or gel composition, can be used. Higher concentrations can be used for sachets, non-abrasive gels and subgingival gels.

Various embodiments may include a humectant, an additive that helps to keep various compositions such as toothpaste from hardening upon exposure to air. Additional benefits from the addition of hemectants include improved moth feel including an enhanced moist feel to the mouth. Some hemectant's may also impart a desirable sweet flavor to various compositions. A typical humectant, on a pure humectant basis, generally comprises from about 0% to about 70%, preferably from about 5% to about 25%, by weight of the compositions herein. Suitable humectants for use in various embodiments include, but are not limited to, edible polyhydric alcohols such as glycerin, sorbitol, xylitol, butylene glycol, polyethylene glycol, and propylene glycol, especially sorbitol and glycerin.

Various embodiments may also include flavoring agents. Suitable flavoring agents for use in various embodiments may include, for example, oil of wintergreen, oil of peppermint, oil of spearmint, clove bud oil, menthol, anethole, methyl salicylate, eucalyptol, 1-menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal known as CGA, and mixtures thereof. Flavoring agents are generally used in the compositions at levels of from about 0.001% to about 5%, by weight of the composition.

Sweetening agents which can be added to various embodiments include, but are not limited to, sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame and cyclamate salts, especially sodium cyclamate and sodium saccharin, and mixtures thereof. A typical composition may include from about 0.1% to about 10% of these agents, in another embodiment from about 0.1% to about 1%, by weight of the composition.

Various embodiments may include coolants, salivating agents, warming agents, numbing agents and analgesics. Typically, agents are included in the compositions at a level of from about 0.001% to about 10%, in another embodiment from about 0.1% to about 1%, by weight of the composition.

Coolants can be any of a wide variety of materials including materials such as carboxamides, menthol, ketals, diols, and mixtures thereof. Various coolants especially useful the present compositions are paramenthan carboxyamide agents such as N-ethyl-p-menthan-3-carboxamide, known commercially as “WS-3”, N,2,3-trimethyl-2-isopropylbutanamide, known as “WS-23,” and mixtures thereof. Additional useful coolants may be selected from the group consisting of menthol, 3-1-menthoxypropane-1,2-di- ol known as TK-10 manufactured by Takasago, menthone glycerol acetal known as MGA manufactured by Haarmann and Reimer, and menthyl lactate known as Frescolat™ manufactured by Haarmann and Reimer. The terms menthol and menthyl as used herein include dextro- and levorotatory isomers of these compounds and racemic mixtures thereof. TK-10 is described in U.S. Pat. No. 4,459,425, Amano et al., issued Jul. 10, 1984. WS-3 and other agents are described in U.S. Pat. No. 4,136,163, Watson, et al., issued Jan. 23, 1979; the disclosures of both are herein incorporated by reference in their entirety.

Salivating agents that may be added to various embodiments include Jambu™ manufactured by Takasago. Typical warming agents that may be added include, for example, capsicum and nicotinate esters, such as benzyl nicotinate. Preferred numbing agents include benzocaine, lidocaine, clove bud oil, and ethanol.

Various embodiments may include an anticalculus agent, for example, a pyrophosphate ion source from a pyrophosphate salt. The pyrophosphate salts useful in the present compositions include the dialkali metal pyrophosphate salts, tetraalkali metal pyrophosphate salts, and mixtures thereof. Disodium dihydrogen pyrophosphate (Na.sub.2H.sub.2P.sub.2O.sub.7), tetrasodium pyrophosphate (Na.sub.4P.sub.2O.sub.7), and tetrapotassium pyrophosphate (K.sub.4P.sub.2O.sub.7) in their unhydrated as well as hydrated forms are the preferred species. In various embodiments at least one pyrophosphate salt may be present in one of three ways: predominately dissolved, predominately undissolved, or a mixture of dissolved and undissolved pyrophosphate.

Compositions comprising predominately dissolved pyrophosphate refer to compositions where at least one pyrophosphate ion source is in an amount sufficient to provide at least about 1.0% free pyrophosphate ions. The amount of free pyrophosphate ions may range from about 1% to about 15%, in another embodiment from about 1.5% to about 10%, and in still another embodiment from about 2% to about 6%. Free pyrophosphate ions may be present in a variety of protonated states depending on the pH of the composition.

Compositions comprising predominately undissolved pyrophosphate commonly refer to compositions that include no more than about 20% of the total pyrophosphate salt dissolved in the composition, preferably less than about 10% of the total pyrophosphate dissolved in the composition. Tetrasodium pyrophosphate salt is the preferred pyrophosphate salt in these compositions. Tetrasodium pyrophosphate may be the anhydrous salt form or the decahydrate form, or any other species stable in solid form in the dentifrice compositions. The salt is in its solid particle form, may be in its crystalline and/or amorphous state, with the particle size of the salt preferably being small enough to be aesthetically acceptable and readily soluble during use. The amount of pyrophosphate salt useful in making these compositions is any amount effective to help control tartar; these amounts generally ranges from about 1.5% to about 15%, in another embodiment from about 2% to about 10%, and in still another embodiment the amount ranges from about 3% to about 8%, by weight of the dentifrice composition. Various embodiments may also include a mixture of dissolved and undissolved pyrophosphate salts. Any of the aforementioned pyrophosphate salts may be used.

Various pyrophosphate salts are described in more detail in Kirk & Othmer, Encyclopedia of Chemical Technology, Third Edition, Volume 17, Wiley-Interscience Publishers (1982), incorporated herein by reference in its entirety, including all references incorporated therein into Kirk & Othmer.

Optional agents to be used in place of or in combination with the pyrophosphate salt include materials such as synthetic anionic polymers, including polyacrylates and copolymers of maleic anhydride or acid and methyl vinyl ether (e.g., Gantrez), as described, for example, in U.S. Pat. No. 4,627,977, to Gaffar et al., the disclosure of which is incorporated herein by reference in its entirety; as well as, e.g., polyamino propoane sulfonic acid (AMPS), zinc citrate trihydrate, polyphosphates (e.g., tripolyphosphate; hexametaphosphate), diphosphonates (e.g., EHDP; AHP), polypeptides (such as polyaspartic and polyglutamic acids), and mixtures thereof.

Various embodiments may also include alkali metal bicarbonate salts. Typically, alkali metal bicarbonate salts may be soluble in water and unless stabilized, they tend to release carbon dioxide in an aqueous system. Sodium bicarbonate, also known as baking soda, is an alkali metal bicarbonate salt commonly used in compositions intended for use oral hygiene and medicines. Various embodiments may include at least one alkali metal bicarbonate salt in a range from about 0.5% to about 30%, or in a range of from about 0.5% to about 15%, and in some cases in a range from about 0.5% to about 5% of the weight of the composition.

Water employed in the preparation of commercially suitable oral compositions should preferably be of low ion content and free of organic impurities. Water generally comprises from about 5% to about 70%, and in another embodiment from about 20% to about 50%, by weight of the composition herein. These amounts of water include the free water which is added plus that which is introduced with other materials, such as with sorbitol.

Titanium dioxide may also be added to the present composition. Titanium dioxide is a white powder which adds opacity to the compositions. Titanium dioxide generally comprises from about 0.25% to about 5% by weight of the dentifrice compositions.

Antimicrobial antiplaque agents may also by optionally present in oral compositions. Such agents may include, but are not limited to, triclosan, 5-chloro-2-(2,4-dichlorophenoxy)-phenol, as described in The Merck Index, 11th ed. (1989), pp. 1529 (entry no. 9573) in U.S. Pat. No. 3,506,720, and in European Patent Application No. 0,251,591 of Beecham Group, PLC, published Jan. 7, 1988; chlorhexidine (Merck Index, no. 2090), alexidine (Merck Index, no. 222; hexetidine (Merck Index, no. 4624); sanguinarine (Merck Index, no. 8320); benzalkonium chloride (Merck Index, no. 1066); salicylanilide (Merck Index, no. 8299); domiphen bromide (Merck Index, no. 3411); cetylpyridinium chloride (CPC) (Merck Index, no. 2024; tetradecylpyridinium chloride (TPC); N-tetradecyl-4-ethylpyridinium chloride (TDEPC); octenidine; delmopinol, octapinol, and other piperidino derivatives; nicin preparations; zinc/stannous ion agents; antibiotics such as augmentin, amoxicillin, tetracycline, doxycycline, minocycline, and metronidazole; and analogs and salts of the above antimicrobial antiplaque agents. If present, the antimicrobial antiplaque agents generally comprise from about 0.1% to about 5% by weight of the compositions of the present invention.

Anti-inflammatory agents may also be present in the oral compositions of the present invention. Such agents may include, but are not limited to, non-steroidal anti-inflammatory agents such as aspirin, ketorolac, flurbiprofen, ibuprofen, naproxen, indomethacin, aspirin, ketoprofen, piroxicam and meclofenamic acid, and mixtures thereof. If present, the anti-inflammatory agents generally comprise from about 0.001% to about 5% by weight of the compositions of the present invention. Ketorolac is described in U.S. Pat. No. 5,626,838, issued May 6, 1997, incorporated herein by reference in its entirety.

Other optional agents include synthetic anionic polymeric polycarboxylates being employed in the form of their free acids or partially or fully neutralized water soluble alkali metal (e.g. potassium and preferably sodium) or ammonium salts and are disclosed in U.S. Pat. No. 4,152,420 to Gaffar, U.S. Pat. No. 3,956,480 to Dichter et al., U.S. Pat. No. 4,138,477 to Gaffar, U.S. Pat. No. 4,183,914 to Gaffar et al., and U.S. Pat. No. 4,906,456 to Gaffar et al., all of which are incorporated herein by reference in their entirety. Typical ratios are about 1:4 to 4:1 copolymers of maleic anhydride or acid with another polymerizable ethylenically unsaturated monomer, including methyl vinyl ether (methoxyethylene) having a molecular weight (M.W.) of about 30,000 to about 1,000,000. These copolymers are available for example as Gantrez (AN 139 (M.W. 500,000), A.N. 119 (M.W. 250,000) and preferably S-97 Pharmaceutical Grade (M.W. 70,000), of GAF Corporation.

Some embodiments selectively include H-2 antagonists including compounds disclosed in U.S. Pat. No. 5,294,433, Singer et al., issued Mar. 15, 1994, which is herein incorporated by reference in its entirety.

Again, at least in part because to their large and uniform surface area the niobium oxides made in accordance with some embodiments of the invention, are useful as coatings in various medical devices, where it is important to promote and intimate contact between the medical devices and, for example, various bone structures. In such applications, they would be readily used in the coating or constructions of screws, clamps, bolts, staples, plates, pins, bars, straps and the like. The presence of niobium oxide nanostructures made in accordance with various embodiments of this invention and the surface of these devices and its inherent ability to react with hydroxyl appetite will promote the formation of strong bonds between the implanted device and the surrounding bone tissue. They may find adventitious use in the treatment of diseased, destroyed, damaged, malformed or missing bone and/or components of teeth.

Niobium oxide nanostructures in accordance with various embodiments of the invention are remarkably uniform and can be readily made in a variety of different surface areas by adjusting perimeters such as electrolyte strength, ionic strength, temperature, potential difference, etc. according to various embodiments of the invention. Niobium oxides made in accordance with various embodiments can have a huge, relatively uniform surface area and they are stable at high temperatures, these physical properties increase their utility in applications such as high temperature catalysis and gas chromatography. Similarly, the niobium oxide nanostructures may be coated with any of a number of different catalysts and used in chemical reactions that take place in either the gaseous or liquid phase.

Typical tip widths can range from about 30 nm to about 400 nm; other ranges include from about 40 nm to about 300 nm, and from about 40 nm to about 100 nm. Nanocone (nanostructure) heights are theoretically constrained only by the thickness of the starting material. Creating higher nanostructures requires longer anodization times or more vigorous anodization conditions for example, higher voltages, higher electrolyte concentrations, temperature adjustments and the like. Niobium oxide is also soluble in HF(aq.); this tends to limit the height of nanostructures that can be formed in the process, irrespective of the thickness of the starting niobium metal.

Typical niobium oxide nanostructures formed in accordance with various embodiments of the invention have heights in the range of about 4 microns to about 60 microns; another range in nanostructure height is between about 6 to about 50 microns.

Niobium oxides made in accordance with some embodiments of the invention can be milled to desired particle sizes. Various milling processes that can be used to mill the oxide include, but are not limited to, bead milling, hammer milling, grating, grinding, and the like.

The uniform shape of the niobium oxide nanostructures readily lends itself to a variety of uses that require high surface area and uniform shape. For example, the niobium oxide nanostructures may be used in the production of sensors in which niobium oxide interacts with at least one component in a sample mixture of gases or liquids. In still another embodiment the niobium oxide nanostructure is coated with a material that selectively interacts with at least one component in a sample of gas or liquid.

In one embodiment the nanostructures of the current invention are coated with materials that hybridize to specific sequences of DNA.

In still another embodiment the nanostructures are coated with materials that bind to tags or labels placed on targeted DNA molecules. Such sensors can be used in the identification, quantification or separation of specific DNA sequences in a given sample. Still other embodiments include niobium oxide nanostructures derivatized or coated with materials such that they differentially interact with bio-molecules including, but not limited to, RNA, polysaccharides, polypeptides, signaling molecules, cell surface markers, hormones, pathogenic organisms, cancer cells and the like.

In one embodiment the niobium oxide nanocones are modified or coated with a material that changes fluorescence when it contact certain nucleic acid polymers such as DNA or RNA. This signal can be detected and use to monitor the presence and/or amount of DNA and/or RNA in a given sample.

Niobium oxides nanostructures can be used in the construction of chromatographic device, for example in gas chromatography or liquid chromatography columns. In some embodiments the niobium oxide may selectively interacts with components of the mixture. Alternatively, niobium oxide nanostructures can be coated with material or that selectively interact with various components of the mixtures. Such devices can be used separation various components in a mixture of compounds.

Niobium oxide nanostructures disclosed in various embodiments can be used in catalyst construction. For example, the surfaces of niobium oxide nanostructures coated with catalysts, increase the reaction rate of reactants contacted with the catalytic surfaces.

Various catalysts that can be coated or layered onto the niobium oxide nanostructures include, but are not limited to precious metals catalysts such as palladium, platinum and the like. Similarly, the niobium oxides may be coated with any of a number of different catalysts and used in chemical reactions that take place in either the gaseous or liquid phase.

### EXPERIMENTAL

For the purpose of promoting further understanding and appreciation of the present invention and its advantages, the following examples are provided. It will be understood, however, that these examples are illustrative and not limiting in any fashion.

### Experiment 1

A section of 99.8% pure niobium foil 0.25 mm thick was purchased from Aldrich and HF acid (48% by assay) was obtained from Fisher Scientific. The niobium metal was rinsed with acetone and ethanol and cut into one centimeter wide strips and the acid was diluted with appropriate amounts of deionized water to achieve 1.5 and 2.5 wt. % concentration. A schematic of the electrochemical anodization system used can be found in FIG. 1.

The electrochemical process is driven by a Sorensen DLM 300-2 power supply that connects to copper and niobium metal electrodes. Contained in a Nalgene 130 mL beaker, the electrodes extend partially into the magnetically agitated electrolyte. The anodization process of the niobium metal was performed under a constant voltage of 25 V at a constant temperature of 22° C.

Secondary electron images were collected using a JEOL JSM-5310LV Scanning Electron Microscope. Diffracted x-rays were collected on Siemens 5000 automated powder diffractometer. Bruker EVA software was then used to fingerprint the diffraction pattern and identify the composition of the material.

### Results

The resulting oxide film formed on niobium metal had a slight light bluish tint while the underlying metal was a smooth, dull gray color. Referring now to FIG. 4 a representative micrograph showing a top view image of niobium oxide anodized for 7.5 hours in 1.5 wt. % HF(aq) electrolyte. The shape is roughly circular, with distortions presumably caused by a combination of grain boundaries and defects in niobium metal along with competitive growth by surrounding neighbors. The size of the single niobium oxide nanostructure in the image is approximately 50 μm; however, structures were found to vary between about 10 and 55 μm within the plane of the oxide film. Visual inspection of the micrograph reveals the prevalence of micro-channels and gaps along the coarse oxide terrain as well as sub-micron sized dendritic-like fingers near the boundary.

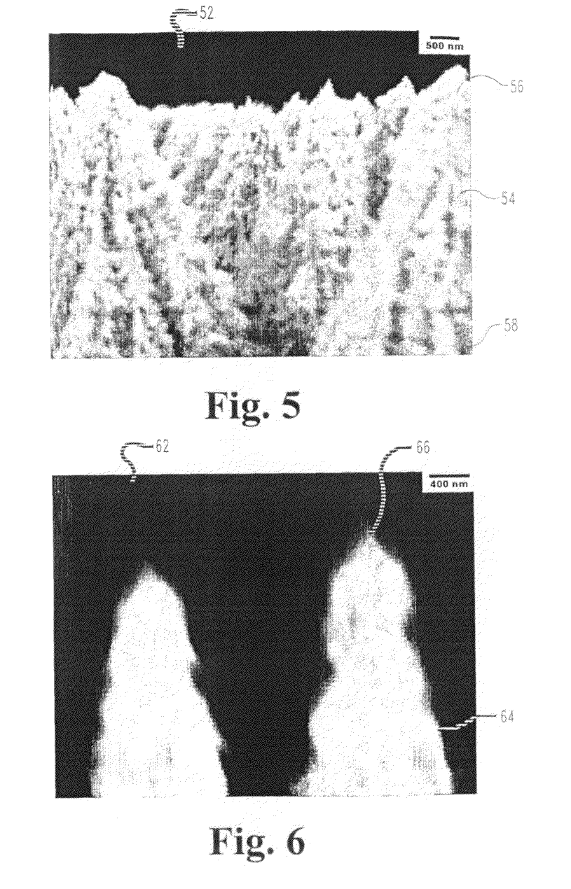

The image of FIG. 5 captures a cross-sectional view (52) of niobium oxide nanostructures (56) formed by anodizing niobium metal under 25 volts in 2.5 wt. % HF for 30 minutes. The resulting nanostructures resemble snow-covered Evergreen trees (54) with heights approximately between 40 and 45 μm and tips (56) ranging between 100 and 300 nm. Anodizing for longer times produces finer tips (66) with reduced sizes less than 50 nm (FIG. 6). Apparently, the coarse terrain observed in FIG. 4 runs axially along the conical nanostructure. Similar architectures to the ones presently discussed were also observed when variations in electrolyte concentration (e.g. 0.25-2.5 wt. % HF) and potential (e.g. 10-90 Volts) were made.

One possible mechanism for this reaction is that it follows the Cabrera-Mott theory [2], where evolution of Nb from bulk metal to surface interacts with adsorbed O₂ or H₂O to form an oxide. If this hypothesis is correct the conical nanostructures may form due to a pronounced expansion in volume upon the formation of Nb₂O₅, which has a volume almost a factor of 3 greater than the volume of the starting material substantially pure niobium metal. As a result, the oxide develops and extends away from the plane of the metal. Evidence that the niobium oxide is Nb₂O₅ can be found in the fact that the diffraction pattern of the material formed in this experiment matches standard X-ray diffraction pattern (card no. 00-030-0873) for Nb₂O₅.

### Experiment 2

A section of 99.8% pure niobium foil 0.25 mm thick was purchased from SIGMA-ALDRICH; Hydrofluoric acid (HF) (48% assay) was obtained from FISHER SCIENTIFIC. The niobium metal was rinsed with acetone and ethanol and cut into one centimeter wide strips and the acid was diluted with appropriate amounts of deionized water to achieve the desired HF wt. % concentrations. The electrochemical process is driven by a SORENSEN™ DLM 300-2 power supply connected to copper and niobium metal electrodes. Potentials of 0 to 40 V were employed 5 to stimulate oxide development. Contained in a Nalgene 100 mL beaker, the electrodes extend partially into the magnetically agitated electrolyte.

Secondary electron images and energy dispersive spectra (EDS) were collected using a JEOL JSM-5310LV Scanning Electron Microscope. Diffracted x-rays were collected on Siemens 5000 automated powder diffractometer. Bruker EVA software was subsequently utilized in fingerprinting the diffraction pattern.

### Results

Referring now to FIG. 7, nanocones comprised substantially of niobium oxide. This cross-sectional view (70) shows the self-organized oxide nanostructure formed by anodizing niobium metal. Anodization conditions include a constant potential of 25 volts for 2 hours in the presence of an electrolyte including about 2.5 wt. % HF.

Bold reflections (74) at the apex of the nanostructures (71) suggest the presence of sub-micron sized tips, while the striations (79) oriented axially along the cones prominently indicate growth orthogonal to the plane of the metal. Since the metal was not annealed prior to experimentation the presence of grain boundaries and defects likely influences the number, size, and origin of the oxide cones as seen in FIG. 7.

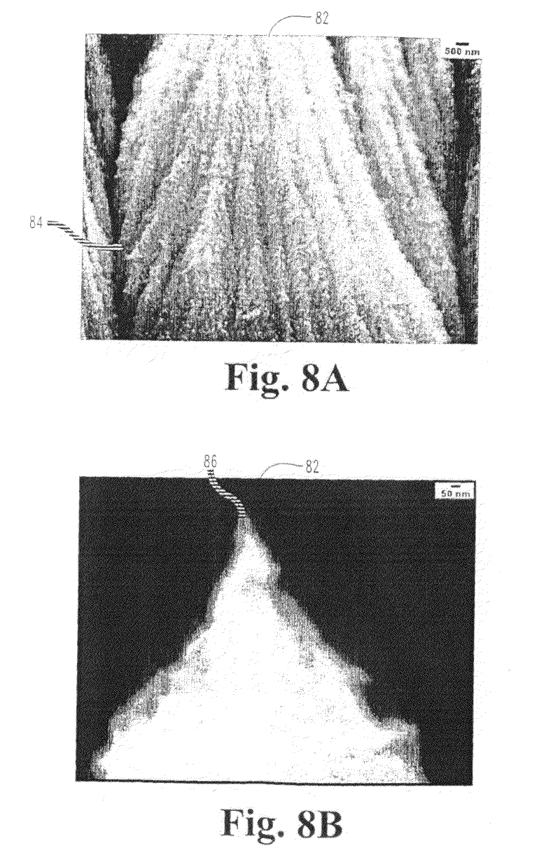

Referring now to FIG. 8(A) a SEM image (82) (side view) of still another embodiment, a niobium oxide microstructure (84) made by anodization. This nanostructure was formed after 2 hours at constant potential of 25 volts in the presence of an electrolyte including about 2.5 wt % HF.

Referring now to FIG. 8(B), close-up micrographs of the conical nanostructures are shown in FIG. 8(A) reveals nanoscale roughness and shallow oxide grooves less than 200 nm wide. Still referring to FIG. 8(B), at the apex (86) of the nanostructure (84), the growth converges to a fine point. Typically the point size varies between 40 and 100 nm when it is formed at standard temperature. At temperatures up to 60° C. the tips became blunt, swelling the tips to sizes up to 300 nm. Regardless of the temperature or time, however, the tips are delicate and fracture easily. Metallic (e.g. AuPd) coatings appear to enhance the integrity of the tips, as well as the cone body. Such stabilization may render these oxide nanostructures as promising templates for applications requiring a fine point source.

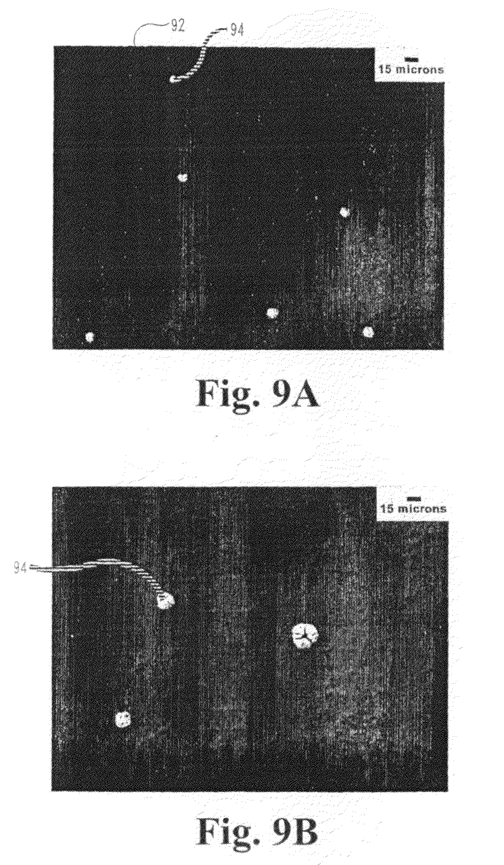

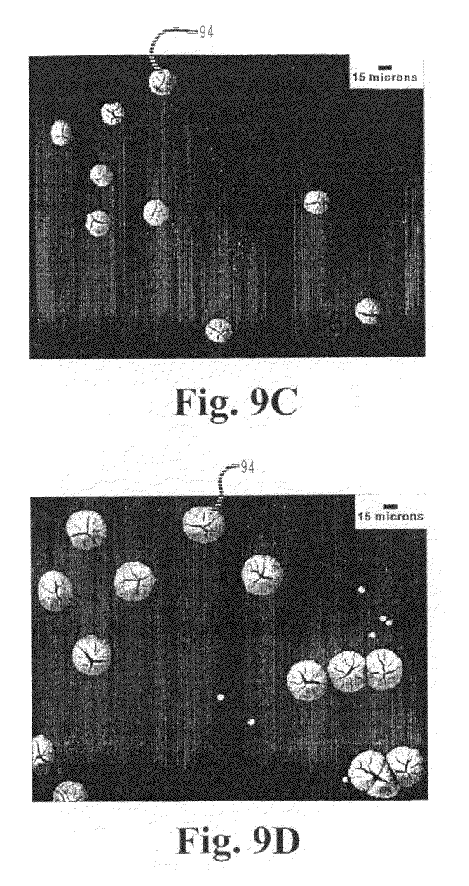

Within the concentration range studied here (0.25-2.5 wt. % HF) the minimum potential required to produce nanocones within one hour at standard temperature and pressure was observed to be 15 V, below which chemical etching of the native oxide occurred. Referring now to FIG. 9 the progression of oxide growth progresses under 15 V and 1.5 wt. % HF (aq) was examined in order to probe the dynamics of microcone (94) growth. Not only do the individual cones (94) augment in size, but the population increases as well, and similar behavior was observed when variations in potentials and electrolyte concentrations were made. Under the present conditions, the in-plane growth rate is approximately two microns per hour while the out-of-plane rate was calculated to be about five microns per hour.

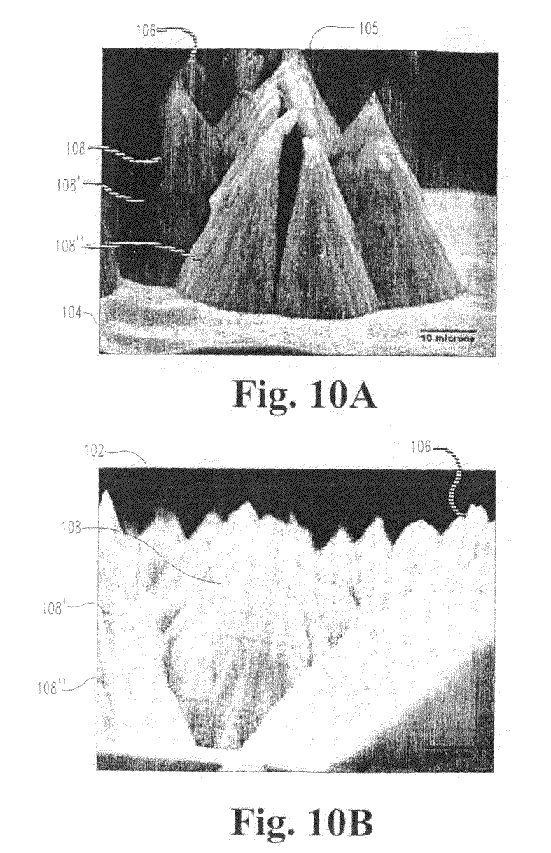

Referring now to FIG. 10, a determination of the kinetics of out-of-plane growth was performed by interrupting the anodization process every hour and counting the resulting ‘rings’ (108), (108′) and (108″). The disparate rates no doubt contribute to the conical shape of the oxide (102). Reducing the temperature did not improve conical shape or texture, but only slowed growth dynamics.

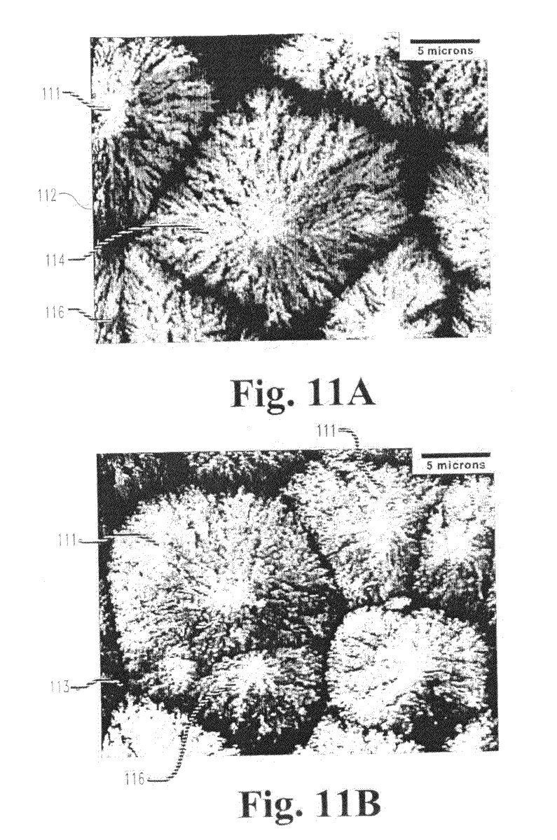

Referring now to FIG. 9, as image (92) illustrates nanocones (94) which develop at 15 volts appear to be split open as niobium oxide microcone growth progresses. This observation occurs within two hours of anodization and proceeds to dominate all of the structures within a seven hour period. At higher potentials, however, this is not the case as seen in FIGS. 7 and 11. Since the anodic oxide films are produced under potentiostatic conditions, the field strength diminishes as the oxide layer becomes thicker, thereby limiting oxide growth. In addition, oxide development is further impeded by its solubility in HF(aq).

A possible explanation for this effect is that the integrity of the oxide produced at a field strength of 15 V cannot compete with the dissolution rate of the oxide. Presumably, the result of such competition is manifested as split pinnacles and conical body gaps in the oxide morphology as shown in FIG. 9.

By fixing the concentration of HF and increasing the potential to 30 and 40 V (FIG. 11) the absence of tears and gaps in the morphology of the nanostructures (112) indicates that oxide formation is favored at higher field strengths and dominates oxide dissolution. Despite the relatively intact structures widespread oxidation at higher potentials introduces crowding (113), thereby constraining in-plane growth and affecting the overall morphology of the cones as observed in image (113).

EDS and X-Ray Diffraction confirmed the oxides formed in the present study are Nb₂O₅ (card no. 00-030-0873). These results are in agreement with published results depicting Nb₂O₅ as the most stable of the niobium oxides [2,14]. The fact that Nb₂O₅ is formed may help to explain the shape of the oxide; as the volume expands by nearly a factor of three relative to the volume of the niobium metal used in the process.

In order to effectively relieve the induced strain due to Nb₂O₅ formation, the resulting oxide nanostructures must protrude from the plane of the metal. Additionally, because there are fewer steric constraints orthogonal to the plane of the metal as discussed above, the growth rate can be expected to be faster in this direction. Therefore, it is possible that the asymmetric growth rates influence the conical shape of the nanobodies.

Using this process nanocones with nanometer-sized tips were prepared by anodizing niobium in HF(aq) electrolyte at standard temperature and pressure. The oxide identified as Nb₂O₅ and the dimensions and integrity of the cones were found to vary with potential, electrolyte concentration, temperature, and anodization time. Fine tips between 40 and 100 nm were readily achievable with sufficiently long anodization times under standard temperature and pressure.

At standard temperature and pressure the development of niobium oxide has been experimentally observed with XPS. It may be possible to rationalize these results in terms of a Cabrera-Mott process [2]. Without being bound by any theory it may be that adsorbed O₂ and H₂O react with conduction electrons of the metal (e.g. Nb) to produce O₂—, the kinetics of which become especially enhanced under wet conditions as in the study presented here [1]. Once ionized, O₂-diffuses into the metal via grain boundaries and defects to react with Nb ions and form the oxide.

### Experiment 3

A sample of niobium oxide was formed by anodization of substantially pure niobium metal in the presence of an electrolyte that included 2.5% HF (aq) and 100 mg of NaF per 100 ml. The anodization was carried out at 46 degrees C. for 68 minutes. The crystalline niobium oxide was soaked in a solution of artificial saliva for 16 hours. Referring now to FIG. 12, the X-Ray Diffraction pattern (120) of the material after it was immersed in artificial saliva. The pattern (122) has a feature (124) marked with an asterisk which is characteristic of HAP this feature matches the standard for HAP JCPDS # 09-0432.

A sample of niobium oxide was formed by anodization of substantially pure niobium metal in the presence of an electrolyte that included 2.5% HF (aq) but no NaF. The anodization was carried out at 46 degrees C. for 2 hours. The crystalline niobium oxide was soaked in a solution of artificial saliva for 16 hours.

Referring now to FIG. 13, the X-Ray Diffraction pattern (131, solid line) of the oxide formed in the presence of NaF and soaked in artificial saliva was plotted on the same graph as the X-Ray Diffraction pattern (133, broken line) of the oxide formed in the absence of NaF and also immersed in artificial saliva. Both patterns (131 and 130) have the features characteristic of Nb₂O₅ and matched well with the standard pattern for this compound (JCPDS # 30-0873). However, only the pattern (131) of the oxide formed in the presence of NaF had a feature (135) marked with an asterisk that is characteristic of the presence of HAP, Ca₁₀(PO₄)₆(OH)₂.

These results indicate that HAP, a major component of teeth and bone, binds to crystalline niobium oxide formed when niobium metal is anodized in the presence of NaF.

### Experiment 4

A sample of niobium oxide was formed by anodizing substantially pure niobium metal in the presence of an electrolyte that included 2.5% HF(aq). In the first trial the process was run for 90 minutes at temperature of 50 degrees C. in an electrolyte that included 100 mg of NaF per 100 mL, at a constant potential of 20V. Once the crystalline niobium oxide was formed it was immersed in artificial saliva for about 19 hours and the X-Ray Diffraction pattern of the material was determined.

Referring now to FIG. 14(A) SEM image (140) shows that crystalline niobium oxide microcone (141) binds HAP crystal (143).

In the first trial the process was run for 90 minutes at temperature of 46 degrees C. in an electrolyte that included 200 mg of NaF per 100 mL, at a constant potential of 20V. Once the crystalline niobium oxide was formed it was immersed in artificial saliva for about 19 hours and the X-Ray Diffraction pattern of the material was determined. Referring now to FIG. 14(B), SEM image (144) shows that crystalline niobium oxide microcone (144) binds HAP crystal (146).

Images (140) and (142) help to confirm that HAP binds to crystalline niobium oxide formed by anodizing niobium metal in the presence of sodium fluoride (NaF).

All references, patents, patent applications and the like cited herein and not otherwise specifically incorporated by references in their entirety, are hereby incorporated by references in their entirety as if each were separately incorporated by reference in their entirety.

An abstract is included to aid in searching the contents of the application it is not intended to be read as explaining, summarizing or otherwise characterizing or limiting the invention in any way.

While the invention has been illustrated and described in detail, this is to be considered as illustrative, and not restrictive of the patent rights. The reader should understand that only the preferred embodiments have been presented and all changes and modifications that come within the spirit of the invention are included if the following claims or the legal equivalent of these claims.

The present invention contemplates modifications as would occur to those skilled in the art. It is also contemplated that processes embodied in the present invention can be altered, duplicated, combined, or added to other processes as would occur to those skilled in the art without departing from the spirit of the present invention.

Unless specifically identified to the contrary, all terms used herein are used to include their normal and customary terminology.

Further, any theory of operation, proof, or finding stated herein is meant to further enhance understanding of the present invention and is not intended to make the scope of the present invention dependent upon such theory, proof, or finding.

While the invention has been illustrated and described in detail in the drawings and foregoing description, the same is considered to be illustrative and not restrictive in character, it is understood that only the preferred embodiments have been shown and described and that all changes and modifications that come within the spirit of the invention are desired to be protected.

### REFERENCES

1. Kovacs, K., Kiss, G., Stenzel, M., and Zillgen, H. J. Electrochem. Soc. 150 (2003) B361-B366.
2. Halbritter, J. Appl. Phys. A. 43 (1987) 1-28.
3. Velten, D., Eisenbarth, E., Schanne, N., and Breme, J. J. Mat. Sci: Mat. Med. 15 (2004) 457-61.
4. Sieber, I., Hildebrand, H., Friedrich, A., and Schmuki, P. Electrochem. Comm. 7 (2005) 87-100.
5. Lu, Q., Hashimoto, T., Skeldon, P., Thompson, to G. E., Habazaki, H., and Shimizu, K. Electrochem. Solid-State Lett. 8 (2005) B17-B20.
6. J. W. Schultze, M. M. Lohrengel, Electrochimica Acta 45 (2000) 2499-2513.
7. J. Choi, R. B. Wehrspohn, J. Lee, U. Gosele, Electrochimica Acta 49 (2004) 2645-2652.
8. V. Zwilling, E. Darque-Ceretti, A. Boutry-Forveille, D. David, M. Y. Perrin, M. Aucouturier, Surface and Interface Analysis 27 (1999) 629-637.
9. O. Jassensky, F. Muller, U. Gosele, Applied Physics Letters 72 (1998) 1173-1175.
10. D. Gong, C. A. Grimes, O.K. Varghese, W. Hu, R. S. Singh, Z. Chen, E. C. Dickey, Journal of Materials Research 16 (2001) 3331-3334.
11. H. Masuda, K. Fukuda, Science 268 (1995) 1466-1468.
12. F. Keller, M. S. Hunter, D. L. Robinson, Journal of the Electrochemical Society 100 (1953) 411-419.
13. M. Ristic, S. Popovic, S. Music, Materials Letters 58 (2004) 2658-2663.
14. M. Grundner, J. Halbritter, Journal of Applied Physics 51 (1980) 397-405

1. A nanostructure, comprising:
a niobium oxide having a substantially conical nanostructure having a tip and a base wherein the tip is substantially thinner than the base.

2. The nanostructure according to claim 1, wherein the tip of said niobium oxide substantially conical nanostructure is between about 30 nm to about 300 nm thick.

3. (canceled)

4. The nanostructure according to claim 1, wherein the height of said nanostructure ranges from about 4 microns to about 65 microns.

5. The nanostructure according to claim 1, wherein the height of said nanostructure ranges from about 5 microns to about 50 microns.

6. The nanostructure according to claim 1, wherein said niobium oxide conical nanostructure is coated with at least one metal.

7. The nanostructure according to claim 6, wherein said metal is selected from the group consisting of gold, platinum, palladium ruthenium, rhodium, iridium, silver; rhenium, osmium, nickel, copper, zinc and alloys thereof.

8. (canceled)

9. (canceled)

10. (canceled)

11. (canceled)

12. A bioactive material, comprising: substantially pure niobium oxide, wherein of said niobium oxide is formed by anodizing niobium metal in the presence of hydrofluoric acid and at least one salt.

13. The bioactive material according to claim 12, wherein the salt is selected from the group consisting of NaF and Na₂SO₄.

14. The bioactive material according to claim 12, wherein said substantially pure niobium oxide binds calcium hydroxylapatite (HAP).

15. A method of forming bioactive crystalline niobium oxide, comprising the steps of:
providing a portion of niobium metal; and
anodizing said portion of niobium metal in the presence of an electrolyte wherein said electrolyte includes hydrofluoric acid (aq.) and at least one salt.

16. The method according to claim 15, wherein said anodizing step is carried out at a constant voltage.

17. The method according to claim 15, wherein said anodizing step is carried out at a constant voltage of between about 15 volts to about 150 volts.

18. The method according to claim 15, wherein said anodizing step is carried out at a constant voltage of between about 15 volts to about 75 volts.

19. The method according to claim 15, wherein said anodizing step is carried out at a temperature of between about −10 degrees Celsius to about 110 degrees Celsius.

20. The method according to claim 15, wherein said anodizing step is carried out at a temperature of between about 20 degrees Celsius to about 110 degrees Celsius.

21. The method according to claim 15, wherein said anodizing step is carried out at a temperature of between about 20 degrees Celsius to about 90:degrees Celsius.

22. The Method according to claim 15, wherein the electrolyte solution includes a dilute level of hydrofluoric acid.

23. (canceled)

24. The method according to claim 15, wherein the level of hydrofluoric acid present in the electrolyte at the start of the anodization step is between about 0.2 wt. percent to about 15 wt. percent.

25. The method according to claim 15, wherein said salt level in the electrolyte is between about 10 mg of salt per 100 ml of electrolyte to about 350 mg of salt per 100 ml of electrolyte.

26. The method according to claim 15, wherein said salt in the electrolyte is selected from the group consisting of NaF and Na₂SO₄.

27. A method of treating a medical condition, comprising the steps of:
providing a medical device or a therapeutic formulation having at least one surface including bioactive crystalline niobium oxide wherein said niobium oxide is formed by anodizing a portion of niobium metal in the presence of an electrolyte including an amount of hydrofluoric acid(aq.);
contacting said bioactive surface of said device with human or animal structures substantially comprised of hydroxylapatite.

28. The method according to claim 27, wherein said medical device is selected from the group consisting of, screws, plates, rods, staples, bars, plates, pegs, dolls, bands, straps, cords, braces and filings.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. The nanostructure according to claim 1, wherein said niobium oxide nanostructure is formed by anodizing a portion of niobium metal in an electrolyte solution comprising an acid and an electrolyte.

56. The method according to claim 15, further including the step of:
forming a population of niodium oxide particles having a given particle size by a process selected from the group consisting of: milling, grinding, or crushing said niobium oxide.

57. The method according to claim 18, wherein said therapeutic formulation is selected from the group consisting of glues, cements, washes, solutions, pastes, coatings, sprays and packings.
